(19) United States
(12) Patent Application Publication
(10) Pub. No.: US 2013/0261059 A1
(43) Pub. Date: Oct. 3, 2013

(54) Compositions and Methods for the Diagnosis and Treatment of Primary Insulin-Like Growth Factor Deficiency (PIGFD) and Idiopathic Short Stature (ISS)

(71) Applicants: Yair Argon, Wynnewood, PA (US); Adda Grimberg, Narberth, PA (US)

(72) Inventors: Yair Argon, Wynnewood, PA (US); Adda Grimberg, Narberth, PA (US)

(73) Assignee: THE CHILDREN'S HOSPITAL OF PHILADELPHIA, Philadelphia, PA (US)

(21) Appl. No.: 13/699,959

(22) PCT Filed: May 26, 2011
(86) PCT No.: PCT/US11/38174

§ 371 Date: Jun. 11, 2013

(30) Foreign Application Priority Data
May 26, 2010 (US) 61348584

(51) Int. Cl. C12Q 1/68; G01N 33/68; A61K 38/27; G01N 33/74

(52) U.S. Cl. CPC C12Q 1/6883; G01N 33/74; G01N 33/6872; A61K 38/27
USPC 514/11.4; 435/6.11; 506/9; 435/6.13; 435/7.1; 530/397

(57) ABSTRACT

Compositions and methods for identifying patients at increased risk for PIGFD and ISS are disclosed.

This application claims priority to U.S. Provisional Application, 61/348,584 filed May 26, 2010, which is incorporated herein by reference as though set forth in full.

This work was supported in part with grant money from the National Institutes of Health: Grant Number, AG-18001S1. Accordingly, the United States government has rights in the invention described herein.

### FIELD OF THE INVENTION

This invention relates to the fields of pediatrics and growth regulation. More specifically, the invention provides compositions for diagnosing disorders associated with insulin-like growth factor (IGF) deficiency and methods of using such compositions in the treatment of these disorders.

### BACKGROUND OF THE INVENTION

Several publications and patent documents are cited throughout the specification in order to describe the state of the art to which this invention pertains. Each of these citations is incorporated herein by reference as though set forth in full.

Glucose Regulated Protein 94 (GRP94) resides in the endoplasmic reticulum and is a molecular chaperone or stress protein which is a member of the heat shock protein (HSP) 90 family. The family includes the htpG gene in bacteria, HSP82 in yeast, HSP90 α and β in higher eukaryotes and the TRAP 1 protein in mitochondria (Buchner, J. 1999. Trends Biochem Sci 24:136). HSP 90 proteins are ligand regulated and participate in the conformational maturation of protein substrates involved in diverse cellular activities ranging from cell signaling to bacterial recognition and immunomodulation. Extensive work in cell culture models show that GRP94 expression is regulated by reduced levels of glucose (Lee, A. S., et al., J. Biol. Chem., 1983. 258: p. 597-603), perturbations of cellular calcium level (Drummond, I. A., et al., J. Biol. Chem., 1987. 262(26): p. 12801-5; Little, E. and A. S. Lee, J. Biol. Chem., 1995. 270(16): p. 9526-34) or the redox potential (Kim, Y. K., K. S. Kim, and A. S. Lee, J. Cell. Physiol., 1987. 133(3): p. 553-559), inhibition of glycosylation, or activation of the unfolded protein response (Gass, J. N., N. M. Gifford, and J. W. Brewer, J Biol Chem, 2002. 277(50): p. 49047-54).

In childhood, IGF deficiency presents with proportionate growth failure. It has been recognized traditionally as a secondary phenomenon to growth hormone (GH) deficiency because GH is the principal inducer of IGF-I production. However, there has been increasing awareness of primary IGF deficiency, which refers to abnormally low IGF levels despite adequate GH production (1). The cause of primary IGF deficiency remains unknown for most cases. Identifying the underlying defects has garnered greater importance since 2003 when the FDA approved GH therapy for idiopathic short stature, or severe non-GH deficient short stature. This indication is a heterogeneous collection of uncharacterized etiologies, including primary IGF deficiency (1, 2). Since GH therapy is so expensive, and clinical responsiveness to GH is so varied among children with idiopathic short stature, identifying the underlying mechanisms is critical to better guide therapeutic decisions (3).

### SUMMARY OF THE INVENTION

In accordance with the present invention, a method for identifying a subject with an increased risk of primary insulin-like growth factor deficiency is provided. An exemplary method entails obtaining a sample from a patient and determining whether GRP94 is altered relative to the wild type sequence, wherein altered GRP94 is correlated with reduced insulin-like growth factor secretion. Patients having GRP94 alterations exhibiting this feature have an increased risk of PIGFD.

In another aspect of the invention, a method for identifying a subject with an increased risk of idiopathic short stature is provided. An exemplary method entails obtaining a sample from a patient and determining whether GRP94 is altered relative to the wild type sequence, wherein altered GRP94 is correlated with reduced insulin-like growth factor secretion. Patients having GRP94 alterations exhibiting this feature have an increased risk of ISS. In another approach, patients presenting in the clinic with ISS would be tested to ascertain whether a GRP94 genetic alteration is contributing to the phenotype.

In another embodiment of the invention, a method for identifying agents which modulate GRP94 regulation of IGF secretion is provided. An exemplary method entails providing cells expressing a single nucleotide polymorphism selected from the group consisting of those set forth in Table 1 or Table 2 (wherein N636H is excluded), providing cells which express the cognate sequences which lack the polymorphisms and contacting the cell populations with a test agent and analyzing whether said agent alters IGF secretion in cells comprising an altered GRP94 relative to those that do not, thereby identifying agents which modulate GRP94 regulation of IGF secretion. In a preferred embodiment, those GRP94 variations which exhibit no phenotype are excluded from the assays disclosed herein. Agents identified using the aforementioned methods are also encompassed by the present invention.

In another embodiment of the invention, a method for identifying agents which modulate GRP94 phosphorylation is provided. An exemplary method entails providing cells expressing a single nucleotide polymorphism selected from the group consisting of those set forth in Tables 1 or 2 (wherein N636H is excluded), providing cells which express the cognate sequences which lack the polymorphisms and contacting the cell populations with a test agent and analyzing whether said agent alters GRP94 phosphorylation in cells comprising an altered GRP94 relative to those that do not, thereby identifying agents which modulate GRP94 phosphorylation and metabolic function. Agents identified using the aforementioned methods are also encompassed by the present invention.

According to yet another aspect of the present invention, there is provided a method of treating PIGFD and ISS in a patient determined to have at least one prescribed single nucleotide polymorphism indicative of the presence of an GRP94 variation which is associated with altered GRP94 function, as described hereinbelow, by administering to the patient a therapeutically effective amount of at least one agent useful to treat these conditions such as human growth hormone. This method provides a test and treat paradigm, whereby a patient's genetic profile is used to personalize treatment with therapeutics targeted towards specific metabolic defects found in individuals exhibiting PIGFD and ISS. Such a test and treat model may benefit up to 50% of patients with PIGFD and ISS with greater efficacy and fewer side effects than non-personalized treatment.

### BRIEF DESCRIPTION OF THE DRAWINGS

FIG. 1. Schematic structure of the HSP90B1 gene and the relation to the domain structure of its product, the GRP94 protein. A. Exon-intron organization of the gene with exons depicted as blue boxes with their corresponding numbers. Green bars, some of the known HapMap synonymous SNPs. Red bars, the five non-synonymous known SNPs. B. The domains of GRP94 are color coded. Numbers indicate the boundary residues of each domain. Dashed lines indicate the exon encoding the indicated domain. C. Non-synonymous SNPs in GRP94 are shown in red. FIG. 1D. A graph showing survival of serum-deprived MEF cells. Cell lines: WT, wild type GRP94; KO, grp94^{-/-}; KO+WT GRP94: grp94^{-/-} transfected with wild type grp94 gene.

FIG. 2. FIG. 2A. IGF-2 secretion by MEF and COS-1 cell lines. WT GRP94, wild type; Clone 2, grp94^{-/-} MEF transfected with wild type grp94 gene: Empty Vector, grp94^{-/-} expressing empty vector; KO, grp94^{-/}. Similar secretion data for IGF-1 were obtained in 10T1/2 cells. FIG. 2B Enrichment of cells expressing GRP94-mGFP when grown in serum-free medium.

FIG. 3. Co-transfection of GRP94-mCherry with cytosolic GFP demonstrates that cells expressing the former are rescued from death, while cells expressing only GFP die, comparable to untransfected cells (not shown).

FIG. 4. Differential survival of grp94−/− cells in serum-free medium when transiently expressing GRP94-GFP mutants. ΔK is an in-frame deletion of amino acids 144-488 that serves as a chaperone-dead negative control. The point mutation D128N is incapable of binding ATP in vitro (left panel). K513N is the human common SNP in the middle domain, which has reduced activity compared to WT GRP94. Data are a representative experiment out of four for each mutant.

FIG. 5. Diagram of the PCR amplification scheme that will be used to sequence the GRP94 gene in test subjects.

FIG. 6. FIG. 6A. Differential survival of grp94^{-/-} cells in serum-free medium when transiently expressing various GRP94-GFP mutants. Blue, WT; Red, E82A; an ATPase deficient mutant in vitro; Green, the K513N mutant found in SNP Rs34482425. B. hGRP94 variant is a secreted form. 293T cells expressing the exon 18 deletion mutant have lower steady state levels than cells expressing wild type GRP94 (WT). Cells were collected at the indicated time points after transfection. Lysates were analyzed by immunoblotting with 9G10 anti-GRP94 monoclonal antibody. GFP served as transfection and loading control. ex., exogenous GRP94; en., endogenous GRP94. C. Pulse-chase analysis of WT (black circles) and exon18 deletion (black squares). Plotted is the fraction of the total pool of GRP94 found in the culture supernatant. Intracellular proteins like tubulin or BiP were not secreted.

FIG. 7. GRP94 Ser/Thr phosphorylation regulates GRP94 binding. A. Phosphorylated GRP94 does not bind L chains. MOPC315.37 cells were metabolically labeled with ³²P or ³⁵S, as indicated. Lane 1, rat monoclonal anti-GRP94 immuno-precipitation of non-crosslinked lysates. Lanes 2-3, rabbit anti-X immunoprecipitation of crosslinked lysates. The relative number of cells loaded for each line is 1.0× (lane 1), 9.0× (lane 2), 1.5× (lane 3). B. GRP94 is phosphorylated on Ser/Thr residues. ³²P labeled GRP94 was gel-purified, acid-hydrolyzed, and analyzed by two-dimensional thin-layer chromatography. The location of each phosphoamino acid marker is indicated.

### DETAILED DESCRIPTION OF THE INVENTION

Insulin-like growth factors (IGFs) mediate growth in childhood and activate major signaling pathways important for cell survival and proliferation throughout life. The production of IGFs varies between individuals and tissues as well as throughout the lifespan, and is controlled by complex endocrine, paracrine and autocrine interactions. Recent work in our lab has characterized a novel control mechanism of IGF production and secretion—interaction with the chaperone Glucose Regulated Protein 94 (GRP94). This interaction is obligatory, and the biosynthesis of either IGF-I or IGF-II is proportional to the activity of GRP94. We hypothesized that variants of human GRP94 with reduced function should be found among subjects with low IGF-I and could even cause primary IGF deficiency. We thus initiated genomic analysis of human GRP94, aimed at relating allelic variations and mutations to serum IGF levels. To date, we identified three sequence variants. One of them, K513N, is a common SNP that was shown, using a novel cell-based assay to be hypomorphic. A second mutant is deletion of the terminal exon 18, which leads to secretion of GRP94 rather than its normal retention in the endoplasmic reticulum. The third is a point mutation in the second domain, whose functional significance has not yet been assessed.

We hypothesize that GRP94 mutations lead to IGF deficiency. This can lead to clinical trials of recombinant IGF-I (Ipsen's product) and if successful as expected, would lead to a new FDA-approved indication for rhiGF-I treatment. The patient having GRP94 mutations causing IGF deficiency can be treated by giving exogenous growth hormone to “push” the system. This approach would be expected to work in patients having mutations conferring partial activity. The data presented herein provide the means to develop new drugs that target the GRP94/GF secretory mechanism itself, thereby improving secretion of endogenous IGF-I. These drugs can be tested in clinical trials and if successful, should be FDA-approved for the treatment of PIGFD/ISS due to GRP94 mutation.

In summary we propose therapeutic strategies that (1) bypass the GRP94 lesion, (2) try to push through a partially functioning GRP94 lesion, and (3) try to correct the lesion via administration of agents that restore GRP94 function.

### DEFINITIONS

As used herein the term “GRP94 protein” is meant to refer to a molecular chaperone which resides in the endoplasmic reticulum and is also known in the art as gp96, ERp99, and endoplasmin. GRP94 is found only in higher plants and metazoans (Nicchitta (1998) Curr Opin Immunol 10:103-109). Stress proteins such as GRP94 are involved in directing the proper folding and trafficking of newly synthesized proteins and in conferring protection to the cell during conditions of heat shock, oxidative stress, hypoxic/anoxic conditions, nutrient deprivation, other physiological stresses, and disorders or traumas that promote such stress conditions such as, for example, stroke and myocardial infarction. nucleic acids, proteins encoded thereby, or other small molecules.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

As used herein, the term “modulate” means an increase, decrease, or other alteration of any or all chemical and biological activities or properties of a wild-type or mutant GRP94 polypeptide. The term “modulation” as used herein refers to both up-regulation (i.e., activation or stimulation) and down-regulation (i.e. inhibition or suppression) of a response.

The phrase “specifically hybridize” refers to the association between two single stranded nucleic acid molecules of sufficiently complementary sequence to permit such hybridization under predetermined conditions generally used in the art (sometimes termed “substantially complementary”). In particular, the term refers to hybridization of an oligonucleotide with a substantially complementary sequence contained within a single stranded DNA or RNA molecule of the invention, to the substantial exclusion of hybridization of the oligonucleotide with single stranded nucleic acids of non complementary sequence.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single stranded or double stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non complementary nucleotide fragment may be attached to the 5′ or 3′ end of the probe, with the remainder of the probe sequence being complementary to the target strand. Alternatively, non complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single stranded or double stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non complementary nucleotide sequence may be attached to the 5′ end of an otherwise complementary primer. Alternatively, non complementary bases may be interspersed within the oligonucleotide primer sequence, provided that the primer sequence has sufficient complementarity with the sequence of the desired template strand to functionally provide a template primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

Amino acid residues are identified in the present application according to conventional three letter or one letter abbreviations. Amino acid residues described herein are preferred to be in the “L” isomeric form. However, residues in the “D” isomeric form may be substituted for any L amino acid residue, provided the desired properties of the polypeptide are retained. All amino acid residue sequences represented herein conform to the conventional left-to-right amino terminus to carboxy terminus orientation.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule, preferably a mutant GRP94 molecule, such as those described below. Samples may include but are not limited to cells, body fluids, including blood, serum, plasma, urine, saliva, tears, pleural fluid and the like.

### Methods of Using GRP94 Variation for Diagnosing a Propensity for the Development of PIGFD and Idiopathic Short Stature

Recombinant human IGF-1 (rhIGF-1), in either ‘naked’ form or complexed with rhIGF binding protein-3, is available to treat PIGFD, though the latter cannot be used for height-related indications per court order. Growth hormone (rhGH) was approved by the FDA in 2003 for the treatment of ISS. The FDA-approved indication for rhIGF-I has been limited to severe PIGFD. A combined rhIGF-I/rhGH product is currently in clinical trials.

The discovery that mutations in GRP94 are associated with altered IGF production can facilitate the diagnosis and management of PIGFD and ISS. Because rhGH treatment for ISS is very expensive (estimated cost ˜$20,000 per patient per year) and controversial (ISS has been extended to off-label use for less severe cases and is perceived by some as the medicalization and treatment of healthy children seeking height enhancement), insurance companies often deny coverage for rhGH treatment of ISS. Data show that some children with ISS respond well to rhGH treatment while others do not. That is because ISS represents a heterogeneous collection of children with growth failure of likely multiple, yet unidentified causes. Identification of the underlying mechanisms has become an important priority in the field to better target treatment. For example, haploinsufficiency of the gene SHOX was identified as causing about 2-3% of cases of ISS and responding well to rhGH treatment. SHOX haploinsufficiency was approved by the FDA as its own indication for rhGH treatment in 2006. Likewise, only a few molecular defects have been identified among rare patients with PIGFD; most cases remain of unknown etiology. It appears that GRP94 mutation may underlie the pathogenic mechanism of PIGFD/ISS, thus providing great clinical interest in GRP94 diagnostic testing and establishing GRP94 mutation as another FDA-approved indication warranting use of rhGH and/or rhIGF-I treatments. New drugs, designed to increase GRP94 expression and/or activity, may also be developed as a novel therapy for these patients.

### Methods of Using GRP94 SNPS Associated with Altered IGF Secretion for Development of Therapeutic Agents

Since the SNPs identified herein have been associated with the etiology of PIGFD and ISS, methods for identifying agents that modulate the activity of altered GRP94 genes and their encoded products containing such SNPs should result in the generation of efficacious therapeutic agents for the treatment of these conditions.

Chromosome 12 contains GRP94 protein coding regions which provide suitable targets for the rational design of therapeutic agents which modulate their activity. Small peptide molecules corresponding to these regions containing the alterations described herein may be used to advantage in the design of therapeutic agents which effectively modulate the activity of the encoded protein.

Molecular modeling should facilitate the identification of specific organic molecules with capacity to bind to the active site of the proteins encoded by the SNP containing nucleic acids based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested. Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells which have a nonfunctional or altered GRP94 gene. These host cell lines or cells therefore encode GRP94 altered at the polypeptide level. The host cells are then grown in the presence of drug compound to determine if the compound is capable of regulating or reversing the altered responsiveness observed in cells carrying the SNP polymorphism. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the novel DNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1N5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter. In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the GRP94 SNPs of the present invention or functional fragments thereof provide a system in which to screen potential compounds or agents for the ability to modulate IGF secretion. Thus, in one embodiment, the nucleic acid molecules of the invention may be used to create recombinant cell lines for use in assays to identify agents which modulate aspects of IGF secretion and subsequent cellular signaling associated with the development of PIGFD and ISS. Also provided herein are methods to screen for compounds capable of modulating the function of proteins encoded by SNP containing nucleic acids.

Another approach entails the use of phage display libraries engineered to express fragment of the polypeptides encoded by the SNP containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved polypeptide activity or stability or which act as inhibitors, agonists, antagonists, etc. of polypeptide activity. By virtue of the availability of SNP containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of GRP94 SNP containing nucleic acids enables the production of strains of laboratory mice carrying such SNPs of the invention. Transgenic mice expressing the SNPs of the invention provide a model system in which to examine the role of the altered GRP94 protein encoded by the SNP containing nucleic acid in regulation of IGF secretion and progression towards PIGFD and ISS. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that altered GRP94 protein plays in various processes associated with IGF deficiency and progression towards PIGFD and ISS phenotypes. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of GRP94 SNP associated with IGF deficiency containing nucleotide sequences.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of altered GRP94 genes and their expression products is to use isolated SNP containing GRP94 genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing SNP containing GRP94 nucleic acid as a targeted insertional cassette provides means to detect a successful insertion as visualized, for example, by acquisition of immunoreactivity to an antibody immunologically specific for the polypeptide encoded by the SNP containing GRP94 nucleic acid and, therefore, facilitates screening/selection of ES cells with the desired genotype.

As used herein, a knock-in animal is one in which the endogenous murine gene, for example, has been replaced with human SNP containing GRP94 gene of the invention. Such knock-in animals provide an ideal model system for studying altered IGF secretion and the subsequent increased risk for the development of PIGFD.

As used herein, the expression of a SNP containing GRP94 nucleic acid, fragment thereof, or SNP containing GRP94 fusion protein can be targeted in a “tissue specific manner” or “cell type specific manner” using a vector in which nucleic acid sequences encoding all or a portion the GRP94 SNP(s) are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein. The nucleic acid sequence encoding the SNP containing GRP94 of the invention may be operably linked to a variety of different promoter sequences for expression in transgenic animals. Such promoters include, but are not limited to a prion gene promoter such as hamster and mouse Prion promoter (MoPrP), described in U.S. Pat. No. 5,877,399 and in Borchelt et al., Genet. Anal. 13(6) (1996) pages 159-163; a rat neuronal specific enolase promoter, described in U.S. Pat. Nos. 5,612,486, and 5,387,742; a platelet-derived growth factor B gene promoter, described in U.S. Pat. No. 5,811,633; a brain specific dystrophin promoter, described in U.S. Pat. No. 5,849,999; a Thy-1 promoter; a PGK promoter; a CMV promoter, the GRP94 promoter or other promoters active in cells involved in insulin like growth factor secretion.

Methods of use for the transgenic mice of the invention are also provided herein. Transgenic mice into which a nucleic acid containing the GRP94 SNP or its encoded GRP94 protein have been introduced are useful, for example, to develop screening methods to screen therapeutic agents to identify those capable of modulating IGF secretion.

### Pharmaceuticals and Methods of Treatment

The elucidation of the role played by the SNPs described herein in modulating the increased risk for PIGFD and ISS facilitates the development of pharmaceutical compositions useful for treatment and diagnosis of PIGFD and ISS. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, aerosolized, intramuscular, and intraperitoneal routes.

Pharmaceutical compositions that are useful in the methods of the invention may be administered systemically in parenteral, oral solid and liquid formulations, ophthalmic, suppository, aerosol, topical or other similar formulations. In addition to the appropriate therapeutic agent, these pharmaceutical compositions may contain pharmaceutically-acceptable carriers and other ingredients known to enhance and facilitate drug administration. Thus such compositions may optionally contain other components, such as adjuvants, e.g., aqueous suspensions of aluminum and magnesium hydroxides, and/or other pharmaceutically acceptable carriers, such as saline. Other possible formulations, such as nanoparticles, liposomes, resealed erythrocytes, and immunologically based systems may also be used to administer the appropriate agent to a patient according to the methods of the invention. The use of nanoparticles to deliver agents, as well as cell membrane permeable peptide carriers that can be used are described in Crombez et al., Biochemical Society Transactions v35:p 44 (2007).

A pharmaceutically acceptable carrier can contain physiologically acceptable compounds that act, for example, to stabilize the agent and/or increase the absorption of the agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients.

In order to treat an individual having PIGFD or ISS, to alleviate a sign or symptom of these disorders, the agent should be administered in an effective dose. The total treatment dose can be administered to a subject as a fractionated treatment protocol, in which multiple doses are administered over a prolonged period of time. One skilled in the art would know that the amount of agent required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having PIGFD and ISS.

In an individual suffering from PIGFD and ISS, in particular a more severe form of the disease, administration of the agent can be particularly useful when administered in combination, for example, with a conventional agent for treating such a disease, such as with GH therapy. The skilled artisan would administer agent, alone or in combination and would monitor the effectiveness of such treatment using routine methods.

Administration of the pharmaceutical preparation is preferably in an “effective amount” this being sufficient to show benefit to the individual. This amount prevents, alleviates, abates, or otherwise reduces the severity of PIGFD and ISS symptoms in a patient.

The following examples are provided to illustrate certain embodiments of the invention. They are not intended to limit the invention in any way.

### Example I

### Identification of Polymorphisms in GRP94 Associated with PIGFD and ISS

### Human GRP94 SNPs

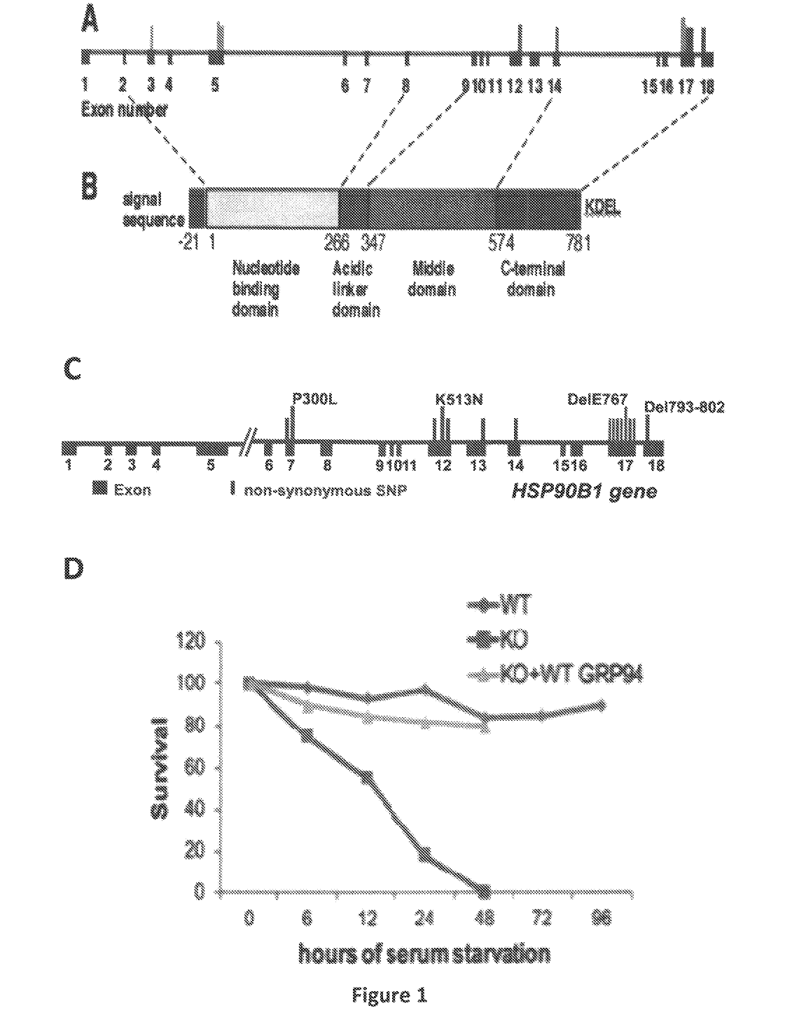

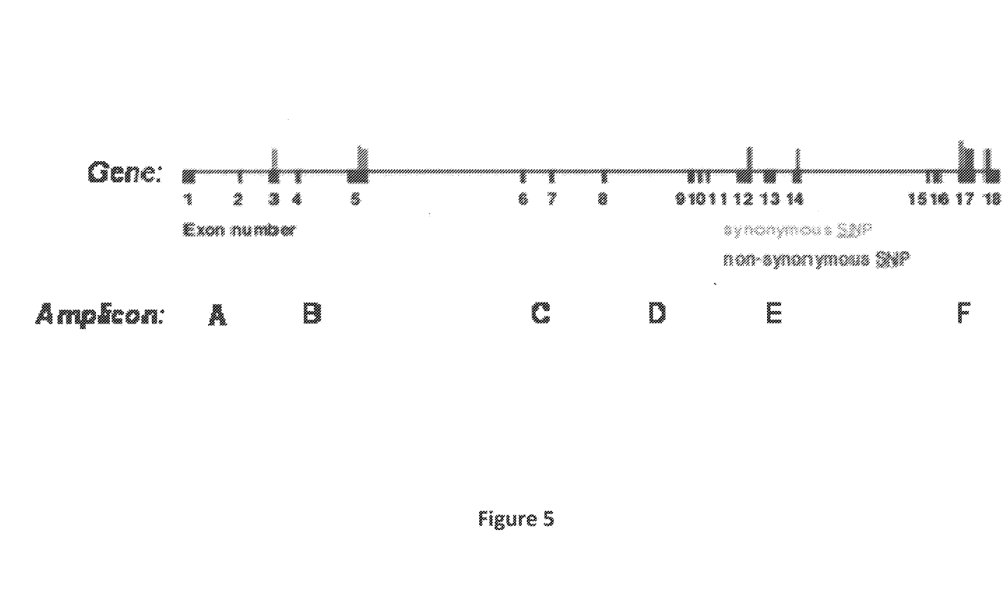

The human HSP90B1 gene, encoding GRP94, is located at chromosome 12q23.3 and consists of 18 exons. See FIGS. 1 and 5. A total of 24 SNPs are known from the HapMap, (18 tag SNPs in Yorubans, 16 in Caucasians), including 4 common SNPs. Five of them are non-synonymous, all in the C terminal half of the protein (red bars, FIG. 1A), where no functional sites have yet been mapped. K513N (rs34482425, exon 12) is found at a frequency of 0.105 in an African American cohort. This SNP is in the M domain and its position in the crystal structure suggests that it is involved in the interaction with the N domain. N636H, D752G, E754V and E781G are the other GRP94 SNPs also present in the C-terminal domain. In addition to common and rare SNPs, at least one rare variant of GRP94 (Ecgp) has been found (16). It has a 17 amino acid insertion at the junction of the N-terminal and acidic linker domains, and therefore this variant may have altered ability hydrolyze ATP, a necessary activity for binding and release of pro-IGF. Thus, variants of GRP94 that could affect its chaperone activity are known to exist in humans. See Tables 1 and 2.

TABLE 1

| Sequence Variation | Source | Phenotype |
|---|---|---|
| Deletion G15 (subsequent | rs34701102 | ND |
| frameshift) | | |
| Missense P300L | IGFD | ND |
| Missense E297K | rs1140570 | ND |
| Missense K513N | rs34482425 | Hypomorphic chaperone |
| Missense E636H | rs3209749 | No activity impact |
| Missense D752G | rs11547718/ | ND |
| | rs7034989 | |
| Deletion E767 | rs5800607 | ND |
| Deletion Exon 18(including | Aging cohort | Secreted |
| KDEL) | | |

TABLE 2

| | | Activity of |
|---|---|---|
| Mutation | Source | GRP94 |
| E82A* | Site-directed by structural prediction | Inactive/ |
| | | no ATPase |
| E297K | rs1140670 HapMap | N.D. |
| P300L | rs116891695 1000 genomes/IGFD patient | Hypomorphic |
| R427A* | Site-directed by structural prediction | Hypomorphic/ |
| | | ATPase |
| | | impaired |
| K442R | Small cell lung carcinoma L55 | N.D. |
| K513N | rs34482425 HapMap | Hypomorphic |
| E527G | rs113326059 Bushmen | N.D. |
| D608A | rs114330942 1000 genomes | N.D. |
| N636H | rs3209749 HapMap | No phenotype |
| A746E | re116946189 1000 genomes | N.D. |
| D752G | rs11547718/rs17034989 | N.D. |
| E754V | rs17159034 HapMap | N.D. |
| E767D | rs5800607 codon deletion/IGFD patient | N.D. |
| E767D | rs117491163 HapMap | N.D. |
| E770K | rs117822561 HapMap | N.D. |
| E781G | rs11547722 1000 genomes | N.D. |
| Del 793-802 | Longevity Consortium | Hypomorphic |
| (exon18) | | |

To sequence human GRP94, we have amplified by PCR the entire coding sequence using 6 amplicons, each including 50-70 bp flanking the exons, and tested the method by sequencing amplicons of three discarded samples, obtained from patients at the Children's Hospital of Philadelphia. As expected, none of the sequences contain somatic mutations. Each DNA sample requires 50 ng and can probably be optimized further. Thus, we have in place the methodology to sequence human hGRP94 variants.

Elucidation of the Functionality of Human GRP94 Variants Using a Cell-Based Assay

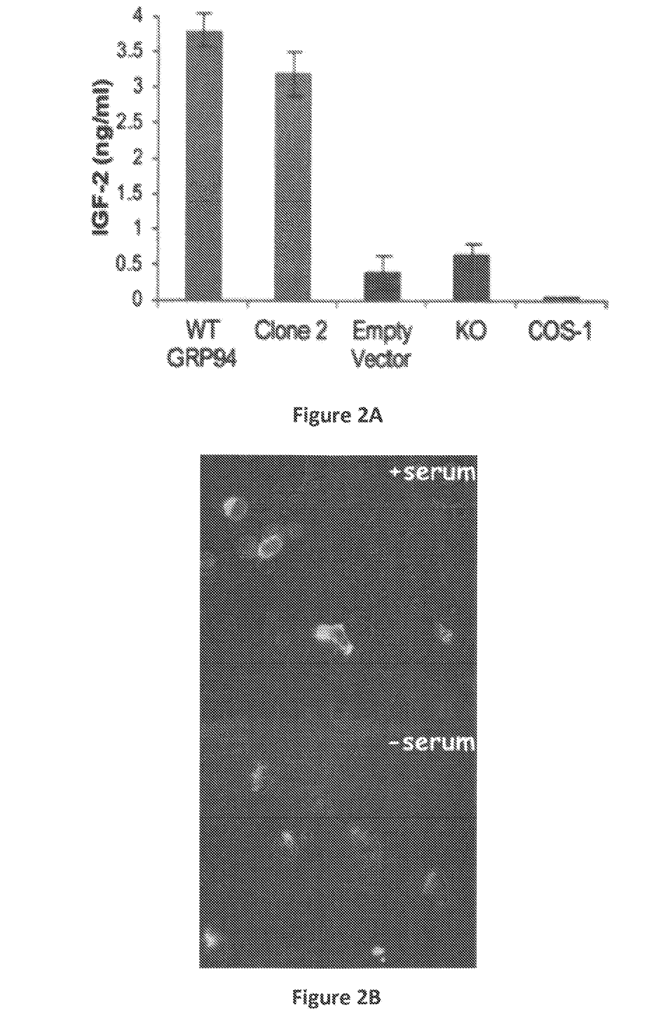

Grp94−/− ES cells, established from knockout murine embryos, grow normally, but are hyper-sensitive to several stress conditions (17, 18). One of these stresses is serum withdrawal and we found that wild type ES cells respond to serum deprivation by initiating the production (and paracrine/autocrine signaling) of IGF-2, while grp94−/− cells are incapable of producing IGF-2 and die by caspase-mediated apoptosis (19). The deficiency in IGF secretion is directly related to GRP94 activity, because when grp94−/− cells are transfected with a grp94 gene, their survival ability and their secretion of IGF-2 are restored (19). Importantly, supplying recombinant IGF also protects the grp94−/− cells from death (19). See FIG. 1D and FIG. 2A.

GRP94 is required not only by IGF-2, but is also needed for the production of IGF-1. In either case the requirement for GRP94 is due to a chaperone-client interaction, without which the pro-hormone fails to advance in its folding to a mature, active hormone (19).

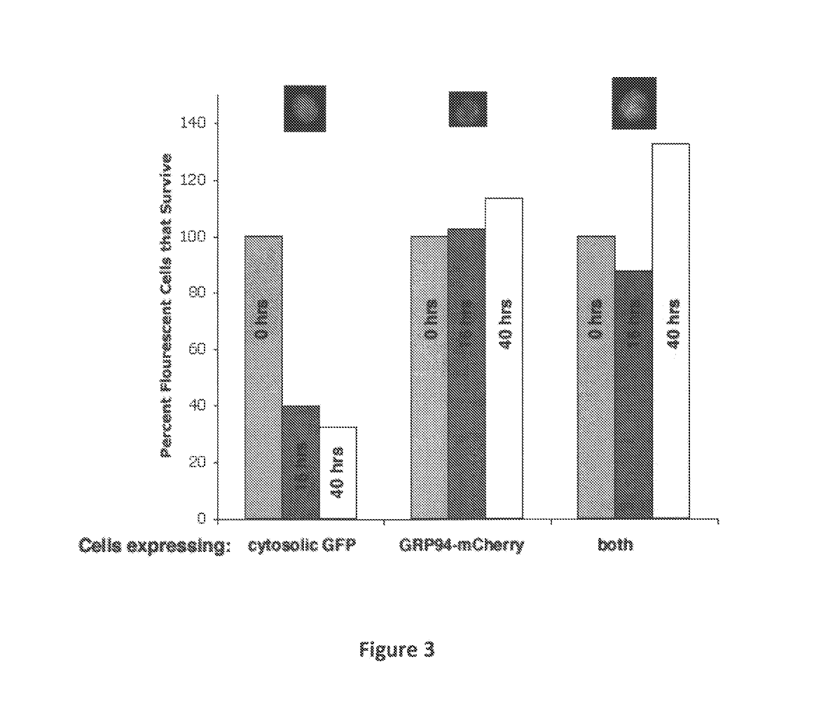

We made fusion proteins of GRP94 with monomeric GFP (FIG. 2B) or monomeric RFP (mCherry, FIG. 3) at the C-terminal end of GRP94, and with a KDEL retention signal, to target the fusion proteins to the ER, like endogenous GRP94. The two fusion proteins are functional, in that they complement the phenotype of grp94−/− cells and only cells expressing the GRP94-fluorescent proteins survive in serum-free medium (FIGS. 2-4). As shown by co-transfection, only cells that express the fusion chaperone, but not untransfected cells or cells expressing cytosolic GFP, survive preferentially.

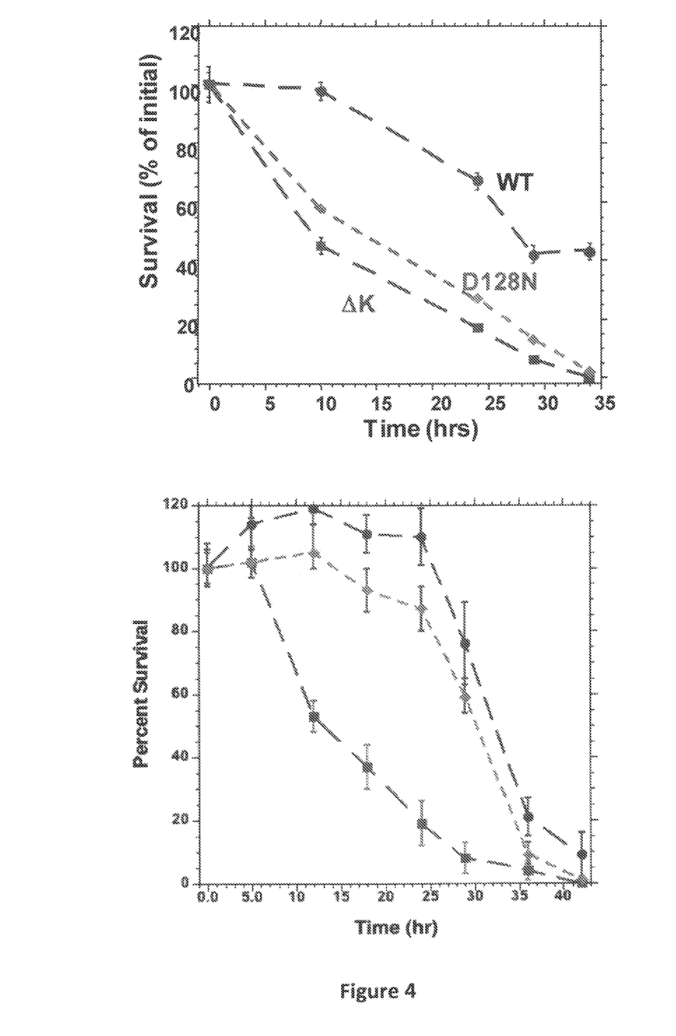

The cell-based assay described above can be used to measure the GRP94-dependent production of IGF (21). See FIG. 2A. We will test sequence variants of hGRP94 and determine whether their expression leads to increased or decreased level of IGF production. Given the informative nature of K513N, we will test each of the variants that are expected to alter either GRP94 folding or its interactions with other proteins, as predicted by mapping the variants onto the crystal structure. As shown in (Ostrovsky et al., 2009b), we used this complementation assay to assess the functionality of a number of GRP94 mutants, including amino acid substitutions that preclude ATP hydrolysis. Importantly, we used this assay to test the K513N substitution, found as a common SNP in Yorubans, and showed it to be a hypomorphic variant. This is proof-of-principle that allelic variations in GRP94 can affect IGF production. We created a chaperone-dead version of GRP94 (red line, FIG. 4) and have mutants that display intermediate activity. This assay has already allowed us to determine the functionality of mutants whose activity had previously only been characterized in vitro.

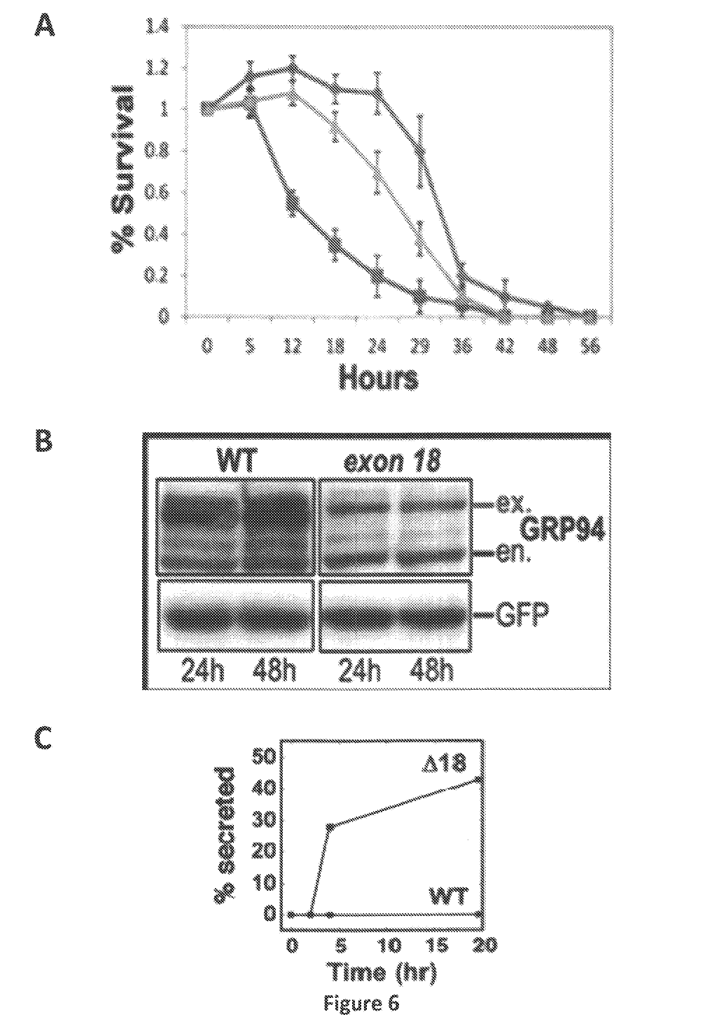

Variation of GRP94 with introduced SNP K513N (green line, FIG. 6) shows diminished activity in IGF-1 production and reduced cell survival in serum free conditions. Moreover, a splicing mutation in GRP94 has been introduced that has been found in >15 subjects in the LonGenity Study cohort from Albert Einstein College of Medicine. This mutation eliminates exon 18 (at the C-terminal tail of the protein) that encodes ER targeting sequence KDEL. The resulting protein is no longer retained in ER, but is instead actively secreted from cells (FIG. 6B+C). This mutant should be less functional in supporting the production of the mature IGF-1. Nine DNA samples from patients with low serum level of IGF-1 have also been sequenced two additional non-synonymous mutants identified. One mutant, Rs60927295, had been previously identified but a novel mutation was also uncovered. P300L is located in the linker domain of GRP94 and could impact the proper folding of the protein and I have already proven that it is less active than WT (Table 3).

TABLE 3

| Variant | Survival Index | N |
|---|---|---|
| WT | 1.00 | 9 |
| E82A | 0.33 ± 0.03 | 9 |
| P300L | 0.71 ± 0.27 | 3 |
| K513N | 0.65 ± 0.12 | 4 |
| R427A | 0.64 ± 0.19 | 4 |

We expect that one or more of the human variants will affect activity in the manner illustrated in FIGS. 4 and 6. It is also conceivable that a sequence variant which increases GRP94 activity can be found, since over-expression of the chaperone is possible and has a positive effect on cell growth and stress survival (Ostrovsky et al., unpublished).

We will apply this assay to assess the functionality of other human GRP94 variants. The advantages of this assay are: 1) a transient transfection experiment (i.e. fast); 2) transfected cells can be visualized directly and tracked live; 3) the level of fluorescence reports on the level of expression per cell.

### Genotyping Children with Extremely Short Stature in Order to Correlate the Genotype with IGF Levels

We suggest that hypomorphic GRP94 variants can cause primary IGF deficiency, and since Rs34482425 has already proven informative, we will genotype children of small stature for the known HapMap SNPs. The five known non-synonymous SNPs (FIGS. 1, 5) are the primary priority, since we seek altered function. Notably, these SNPs are clustered in the Middle and C-terminal domains of the protein and each encodes a non-conservative amino acids substitution. By analogy to related proteins, it is thought that GRP94 binds its client proteins in the middle and/or C domains, so these variants are likely to affect function. Because the crystal structure of GRP94 is known (12) we will be able to interpret amino acids changes that are discovered; we will know whether they are in open faces of the protein or ‘buried’ within the fold, whether they are in proximity to other known sites in the protein (e.g. the dimerization interface in the C domain or the ATPase activation loop in the M domain), etc.

The primary cohort of subjects for genotyping will be from Dr. Ron Rosenfeld (Oregon Health Sciences Center), creator and director of the IGFD Research Center, which collects DNA from patients with severe primary IGFD from all over the world. This repository (>100 children) is a unique resource of clinically rare patients, who have already been characterized auxologically and hormonally. This cohort will be compared to two others that we already have access to: (1) the large genotyping effort in the CHOP Applied Genomic Center, with >3600 children with height and weight measurements (though rarely with IGF measurements). 13 SNPs that tag the GRP94 gene will be compared to the causative SNP in children with short stature (<5 percentile), normal or tall stature (>95 percentile). This population will provide both genotypes for children with diagnosis of short stature (for comparison with the Rosenfeld cohort) and an analysis of the GRP94 SNPs as a quantitative trait to ask if the gene displays association with height/weight in general. The second comparative population is the Health ABC consortium, which is an adult cohort, but includes 623 subjects with longitudinal IGF-1 measurements in addition to height and weight. Genotyping all these subjects will increase considerably the power of the genotype-to-IGF correlation.

We will genotype 100 subjects in Dr. Rosenfeld's cohort, as they have height, weight and IGF-1 data, and analyze all 24 SNPs vs. these parameters. The Health ABC population will be genotyped for these SNPs as well. The CHOP comparative cohort will be analyzed for 13 of these SNPs, already available on the current chip. Because of the size of the gene and the already existing functional SNP (rs34482425) there is high likelihood that additional common functional SNPs will be found. Genotyping will be performed using single SNP analysis with either single base extension/fluorescence polarization (FP) and multiplex SNP analysis (SNP Stream). In consultation with the Core, at least 14 of the SNPs will be genotyped in a multiplex panel and the rest, in singleplex.

Association of SNPs genotype with IGF-1 levels will be tested with linear regression models, assuming a simple additive model. To test the association between rare variants and IGF levels, we will first use a comparison test of rare variants that are expected to be functional (e.g. non-synonymous SNPs) and synonymous SNPs, as described in (20). Statistical tests for the frequency of non-synonymous vs. synonymous alleles in each group will be conducted based on permutation tests.

### Identification of Rare GRP94 Variants

Given that sequence variation in GRP94 is unexplored, there are likely many more polymorphisms than is currently known. Common variants may contribute to the normal variation in human height, and would be expected to occur at higher frequency among shorter individuals (i.e. phenotype less severe than the classic primary IGF deficiency). Rare variants may explain other IGF deficiencies as well as variations in aging, where low IGF is beneficial. To detect such variants, we will sequence candidate subjects from the Rosenfeld cohort and selected control subjects with the highest and lowest 10% IGF-1 level in the Health ABC cohort, where 623 subjects with IGF-1 data are available (359 Whites, 264 Black). We expect to sequence ˜100 subjects from each end of the IGF-1 distribution. The 18 exons of the GRP94 gene will be sequenced using PCR amplicons from genomic DNA samples from those subjects. We are currently able to span the entire gene with 6 short-range PCR amplicons (FIG. 5) and are exploring further improvement in sensitivity. Sequencing will also include the presumed splice sites. The set of primers and conditions that we already validated in 3 patient samples will be used.

### Example 2

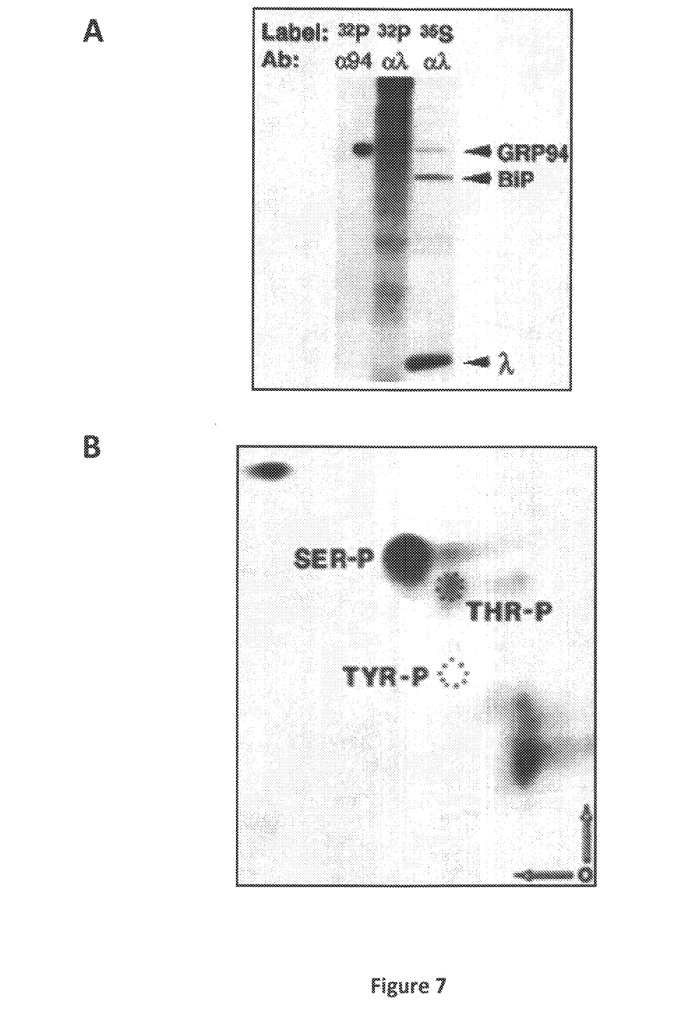

In order to determine whether GRP94 is phosphorylated in vivo, plasmacytoma cells MOCP315.37 and MOCP315.26 were metabolically labeled using ³²P-orthophosphate. Immunoprecipitates of GRP94 revealed that it is phosphorylated (FIG. 7A). Moreover, phosphorylation impacts the association between GRP94 and immunoglobulin light (L) chain, a known client for the chaperone. When GRP94 is phosphorylated, it does not bind to the L chain. This strongly indicates that polypeptide binding of GRP94 is regulated by phosphorylation. In addition, as shown in FIG. 7B, GRP94 is phosphorylated primarily on serine and to a lesser degree on threonine residues. No phosphotyrosine was evident even after long exposure, nor did Western blotting with monoclonal anti-phosphotyrosine antibody reveal a GRP94 phosphorylation (data not shown). Incorporation of ³²P-orthophosphate into GRP94 is a proof that this process happened in live cells with an intact ER compartment.

To further ensure localization of phosphorylated form of GRP94, immunoprecipitates of ³²P labeled GRP94 will be subjected to endo H digestion. While entering the ER compartment, de novo translated GRP94 is N-glycosylated on Asn-196 and thus endo H digestion should produce a shift in the migration of GRP94. To confirm its localization, ER microsomes will be purified by floatation on sucrose gradients; this should be sufficient to determine whether phospho-GRP94 co-purifies with the ER. Finally, to assess semi-quantitatively the fraction of GRP94 that undergoes phosphorylation, the protein will be analyzed by 2-dimensional gels, and the phospho-GRP94 spots with more acidic pI, indicating phosphorylation, will be quantified. To address the functional significance of observed Ser/Thr phosphorylations results showing that the phosphorylated form of GRP94 binds poorly to immunoglobin L chain will be further characterized. The phosphorylation status of GRP94 in cell lines that have dramatically different ER activity will be compared. For example, HepG2, which are considered high “secretors” and 293T cells, which are low level “secretors” will be studied. Second, we will induce the unfolded protein response (UPR) pharmacologically and by over-expression of misfolded proteins in ER. Common inducers of UPR, e.g., thapsigargin and tunicamycin, as well as mutants of α1-anti-trypsin, NHK and NHK-QQQ that do not undergo proteolysis and accumulate in ER will be employed. 293T, HepG2, C2C12 and 42.1 (GRP94+/+ MEFs) cells will be activated in vitro with growth factors (GH, IGF-1, insulin) or cytokines (IL-2, IL-6). After each treatment I will analyze the phosphorylation of GRP94. This approach will be useful in assessing whether the degree of GRP94 phosphorylation changes as the workload of this chaperone, or the metabolic state of the cell, changes. Relevant Ser/Thr sites in GRP94 will also be mapped by LC-MS/MS mass spectrometry.

### REFERENCES

1. Grimberg A, DeLeon D. 2005. Disorders of growth. Philadelphia: Elsevier, Inc. 127-67 pp.
2. Koren D, Grimberg A. 2008. Short Stature. Baltimore: Lippincott Williams & Wilkins. 818-39 pp.
3. Rosenfeld R G. 1996. Biochemical diagnostic strategies in the evaluation of short stature: the diagnosis of insulin-like growth factor deficiency. Horm Res 46: 170-3
4. Kenyon C. 2001. A conserved regulatory system for aging. Cell 105: 165-8
5. Baker J, Liu J P, Robertson E J, Efstratiadis A. 1993. Role of insulin-like growth factors in embryonic and postnatal growth. Cell 75: 73-82
6. Suh Y, Atzmon G, Cho M O, Hwang D, Liu B, Leahy D J, Barzilai N, Cohen P. 2008. Functionally significant insulin-like growth factor I receptor mutations in centenarians. Proc Natl Acad Sci USA
7. Grimberg A. 2003. Mechanisms by which IGF-I may promote cancer. Cancer Biol Ther 2: 630-5
8. Yakar S, Kim H, Zhao H, Toyoshima Y, Pennisi P, Gavrilova O, Leroith D. 2005. The growth hormone-insulin like growth factor axis revisited: lessons from IGF-1 and IGF-1 receptor gene targeting. Pediatr Nephrol 20: 251-4
9. Efstratiadis A. 1998. Genetics of mouse growth. Int. J. Dev. Biol. 42: 955-76
10. Ostrovsky O, Ahmed N T, Argon Y. 2009. GRP94 protects cells grown without serum from apoptotis by regulating the secretion of insulin-like growth factor II. Mol. Biol. Cell 20: 1855-64
11. Schulte T W, Akinaga S, Murakata T, Agatsuma T, Sugimoto S, Nakano H, Lee Y S, Simen B B, Argon Y, Felts S, Toft D O, Neckers L M, Sharma S V. 1999. Interaction of radicicol with members of the heat shock protein 90 family of molecular chaperones. Mol. Endocrinol. 13: 1435-48
12. Dollins D E, Warren J J, Immormino R M, Gewirth D T. 2007. Structures of GRP94-nucleotide complexes reveal mechanistic differences between the hsp90 chaperones. Mol Cell 28: 41-56
13. Immormino R M, Dollins D E, Shaffer P L, Soldano K L, Walker M A, Gewirth D T. 2004. Ligand-induced conformational shift in the N-terminal domain of GRP94, an Hsp90 chaperone. J Biol Chem 279: 46162-71
14. Frey S, Leskovar A, Reinstein J, Buchner J. 2007. The ATPase cycle of the endoplasmic chaperone Grp94. J Biol Chem 282: 35612-20
15. Ostrovsky O, Makarewich C, Snapp E L, Argon Y. 2008. ATP binding and hydrolysis, but not the peptide binding site, are required for the chaperone activity of GRP94 in the cell. Submitted
16. Prasadarao N V, Srivastava P K, Rudrabhatla R S, Kim K S, Huang S H, Sukumaran S K. 2003. Cloning and expression of the Escherichia coli K1 outer membrane protein A receptor, a gp96 homologue. Infect Immun 71: 1680-8
17. Wanderling S, Simen B B, Ostrovsky O, Ahmed N T, Vogen S, Gidalevitz T, Argon Y. 2007. GRP94 is essential for mesoderm induction and muscle development because it regulates IGF secretion. Mol. Biol. Cell 18: 3764-75
18. Biswas C, Ostrovsky O, Makarewich C A, Wanderling S, Gidalevitz T, Argon Y. 2007. The peptide binding activity of GRP94 is regulated by Calcium. Biochem. J. 405: 233-41
19. Ostrovsky O, Ahmed N T, Argon Y. 2007. GRP94 protects cells grown without serum from apoptotis by regulating the secretion of insulin-like growth factor II. J. Cell Biol. in revision
20. Ahituv N, Kavaslar N, Schackwitz W, Ustaszewska A, Martin J, Hebert S, Doelle H, Ersoy B, Kryukov G, Schmidt S, Yosef N, Ruppin E, Sharan R, Vaisse C, Sunyaev S, Dent R, Cohen J, McPherson R, Pennacchio L A. 2007. Medical sequencing at the extremes of human body mass. Am J Hum Genet. 80: 779-91
21. Ostrovsky O, Makarewich C, Snapp E L, Argon Y. 2009. ATP binding and hydrolysis, but not the peptide binding site, are required for the chaperone activity of GRP94 in the cell. Proc Natl Acad Sci USA 106: 11600-11605.

While the invention has been described in detail and with reference to specific examples thereof, it will be apparent to one skilled in the art that various changes and modifications can be made therein without departing from the spirit and scope thereof.

What is claimed is:

1. A method for identifying a subject with an increased risk of primary insulin-like growth factor deficiency, comprising
a) obtaining a sample from said patient and determining whether GRP94 is altered relative to the wild type sequence and exhibits altered GRP94 function, wherein altered GRP94 is correlated with reduced insulin-like growth factor secretion, thereby conferring an increased risk of PIGD.

2. The method of claim 1, wherein said alteration is selected from the alterations shown in Table 1 or Table 2 and excludes N636H.

3. The method of claim 1, wherein said alteration is K513N mutation.

4. A method for identifying a subject with an increased risk of idiopathic short stature, comprising
a) obtaining a sample from said patient and determining whether GRP94 is altered relative to the wild type sequence, wherein altered GRP94 is correlated with reduced insulin-like growth factor secretion, thereby conferring an increased risk of idiopathic short stature.

5. The method of claim 4, wherein said alteration is selected from the alterations shown in Table 1 and excludes N636H.

6. The method of claim 4, wherein said alteration is K513N mutation.

7. A method for identifying agents which modulate GRP94 regulation of IGF secretion comprising,
a) providing cells expressing a single nucleotide polymorphism selected from the group consisting of those set forth in Table 1 or Table 2;
b) providing cells which express the cognate sequences which lack the polymorphisms of step a);
c) contacting the cells of steps a) and b) with a test agent and
d) analyzing whether said agent alters an IGF secretion in cells contacted in step a) relative to those of step b), thereby identifying agents which modulate GRP94 regulation of IGF secretion, with the proviso that GRP94 variations which do not alter GRP94 function are excluded from said method.

8. The method of claim 7, wherein said cells are within an animal which comprises a transgene encoding a GRP94 comprising one or more genetic alterations.

9. An agent identified by the method of claim 7.

10. A method for identifying agents which modulate GRP94 phosphorylation comprising,
a) providing cells expressing a single nucleotide polymorphism selected from the group consisting of those set forth in Table 1 or Table 2;
b) providing cells which express the cognate sequences which lack the polymorphisms of step a);
c) contacting the cells of steps a) and b) with a test agent and
d) analyzing whether said agent alters GRP94 phosphorylation in cells contacted in step a) relative to those of step b), thereby identifying agents which modulate GRP94 phosphorylation, with the proviso that GRP94 variations which do not alter GRP94 function are excluded from said method.

11. A method of treating PIGFD and/or ISS in a human subject determined to have at least one single nucleotide polymorphism (SNP) indicative of the presence of a functionally altered GRP94, said at least one SNP being selected from the group consisting of SNPs set out in Tables 1 and 2, the method comprising administering to said human subject a therapeutically effective amount of at least one agent useful for the treatment of PIGFD and/or ISS.

12. The method of claim 11, wherein said agent is human growth hormone.
